(19) United States
(12) Patent Application Publication
(10) Pub. No.: US 2016/0259913 A1
(43) Pub. Date: Sep. 8, 2016

(54) DRUG DELIVERY DOSE INDICATOR

(71) Applicant: BIOGEN MA, INC., Cambridge, MA (US)

(72) Inventors: Ning YU, Lexington, MA (US); Atul M. PATEL, Lexington, MA (US); Saeed SOKHANVAR, Belmont, MA (US); Michel BRUEHWILER, Newton, MA (US); Cole CONSTANTINEAU, Cambridge, MA (US); Mohammadreza RAMEZANIFARD, Dorchester, MA (US); Daniel YASEVAC, Arlington, MA (US)

(73) Assignee: Biogen MA, Inc.

(21) Appl. No.: 15/057,993

(22) Filed: Mar. 1, 2016

Related U.S. Application Data
(60) Provisional application No. 62/127,005, filed on Mar. 2, 2015.

(51) Int. Cl. G06F 19/00; A61M 5/31

(52) U.S. Cl. CPC G06F 19/3462; A61M 5/31; A61M 2005/3126; A61M 2205/3592; A61M 2205/50; A61M 2205/332; A61M 2205/3331; A61M 2205/3368

(57) ABSTRACT

Embodiments of the disclosure include a transponder for determining a characteristic of a dose delivery device. The transponder may include a first sensor configured to detect a first parameter of the dose delivery device based on movement of at least one of the dose delivery device or a medicament contained within the dose delivery device, wherein the first sensor is configured to generate a first signal indicative of the first parameter. The transponder may include a processor operably coupled to the first sensor and configured to process the first signal. The transponder may include an indicator unit operably coupled to the processor and configured to generate an output based on the first signal, wherein the output is perceivable by a user. The transponder may also include a battery configured to provide power to at least one of the first sensor, the processor, and the indicator unit.

### I. RELATED APPLICATIONS

This patent application claims the benefit of priority under 35 U.S.C. §120 to U.S. Provisional Patent Application No. 62/127,005, filed Mar. 2, 2015, which is incorporated herein by reference.

### I. DESCRIPTION

1. Field of the Disclosure

The present disclosure relates generally to a drug delivery device and, more particularly, to a dose indicator for a drug delivery device.

2. Background of the Disclosure

The development of drug delivery devices has vastly improved the lives of patients who self-administer medication to maintain a treatment schedule. The self-administration of medication provides convenience by reducing the need to report to a physician for regular treatments, but may require the individual or a care provider to inject a prescribed amount of medicament under specific conditions. For example, the medication may need to be administered in a specified location, at a specified angle, at a specific speed, and/or according to a strict schedule. The medicament may also need to be brought to room temperature, mixed, or may require air to be expelled prior to injection. The pain and complexity of the injection may cause anxiety or may decrease compliance, and improper administration of the medicament may reduce efficacy and/or cause other complications.

Controls are typically provided on the delivery devices that allow the operator to select the drug dosage, and an actuator may be activated to dispense the drug. Historically, it was the duty of the operator to report compliance with a treatment regimen to ensure that the dosages were administered as required. However, newer drug delivery devices have been developed with mechanisms that automatically record the administered drug dosages. These mechanisms may be external to the delivery device, and may determine the drug dosage by recording the positioning of the controls indicating the desired dosage.

Although these mechanisms may help to assist the individual in maintaining a drug administration schedule, they may not monitor the actual functioning of the drug delivery device or details of the administration. Specifically, monitoring the controls of the delivery device may not indicate the actual dose of drug being delivered, which may vary due to, for example, malfunctions or user error, including jamming of the plunger, air bubbles present in the drug, and/or blockages of the passageways. These mechanisms also may not be capable of determining critical parameters of the drug delivery, such as the actual pressure applied to the plunger, the speed of injection, and conditions of the drug. Accordingly, available drug delivery systems may not be capable of providing sufficient feedback of the dispensing operation. The disclosed drug delivery device is directed to overcoming one or more of the problems set forth above and/or other problems of the prior art.

### II. SUMMARY OF THE DISCLOSURE

Embodiments of the present disclosure relate to a delivery device, such as a drug delivery device with dose indicator. Various embodiments of the disclosure may include one or more of the following aspects.

In accordance with one embodiment, a transponder for a dose delivery device for determining a characteristic of dose delivery may include a first sensor configured to detect at least a first parameter of the dose delivery device based on movement of at least one of the dose delivery device or a medicament contained within the dose delivery device, wherein the first sensor is configured to generate a first signal indicative of at least the first parameter. The transponder may include a processor operably coupled to the first sensor and configured to process the first signal. The transponder may include an indicator unit operably coupled to the processor and configured to generate an output based on the first signal, wherein the output is perceivable by a user. The transponder may also include a battery configured to provide power to at least one of the first sensor, the processor, and the indicator unit.

Various embodiments of the adaptor may include one or more of the following features: the processor may be configured to determine whether the first parameter falls inside of or outside of a predetermined range; the transponder may be formed as part of the dose delivery device; the transponder may be configured to fit between a push rod and a plunger of a syringe; the transponder may be a stand-alone component dimensioned to fit inside of the dose delivery device; the transponder may also be configured to be coupled to an external portion of the dose delivery device at a location separate from the location of the first sensor; the processor may be configured to communicate with a receiver located external to the transponder; the receiver may be configured to be located separate from the dose delivery device; the receiver may be operably coupled to the indicator unit, and the indicator unit and the receiver may be located at a location different than a location of the first sensor; the output from the indicator unit may be visual; the first parameter may include at least one of a pressure, a speed, an acceleration, a time, a relative displacement, and a position; the first parameter may be indicative of at least one of a start of the delivery, an end of the delivery, or progress of the delivery; the first sensor may include one of a pressure sensor, a strain gauge, and a switch, configured to generate the first signal indicative of a pressure applied to the push rod; the first signal may be generated relative to a threshold; the transponder may also include a second sensor configured to generate a second signal based on a second parameter, wherein the processor is configured to process the second signal in addition to the first signal; the second sensor may include a temperature gauge configured to generate the second signal, and the second signal may be indicative of a temperature of the medicament within the reservoir; and at least one of the first sensor or the second sensor may include at least one of an accelerometer or a gyroscope, configured to determine the relative positioning of the transponder.

In accordance with another embodiment, a dose delivery device having a transponder for determining a characteristic of the dose delivery device when delivering a dose of a medicament. The dose delivery device may include a body housing a reservoir within the body for containing the medicament. The dose delivery device may include an actuator configured for engagement by a user. The dose delivery device may include a push rod coupled to the actuator and extending through the body, the push rod being configured to dispense the medicament from the reservoir. The dose delivery device may include a plunger located distal to the push rod and located adjacent to the reservoir. The transponder may be positioned within the body and may include a sensor configured to detect at least one parameter of the dose delivery device and to generate a signal indicative of the parameter. The transponder may include a processor operably coupled to the sensor and configured to process the signal. The transponder may also include an indicator unit operably coupled to the processor and configured to generate an output based on the signal, wherein the output is perceivable by the user.

Various embodiments of the transponder may include one or more of the following features: the transponder may be positioned within the body at a location proximal the reservoir; the sensor may include at least one of a pressure sensor, a strain gauge, or a switch, and the signal may be indicative of a pressure applied to the push rod; the sensor may include a temperature gauge configured to generate the signal, the signal may be indicative of a temperature of the medicament within the reservoir; the sensor may include one or more of an accelerometer or a gyroscope, configured to determine the relative positioning of the transponder; the dose delivery device may be a syringe; and a distal end of the push rod may abut a proximal end of the sensor.

In accordance with yet another embodiment, a dose delivery system may include a dose delivery device. The dose delivery device may include a body that may have a reservoir located within a distal region of the body, wherein the reservoir may be configured to contain a medicament. The dose delivery device may include a push rod having a plunger located at a distal end of the push rod, wherein the plunger defines a proximal end of the reservoir, and wherein the push rod and the plunger are cooperatively configured to dispense the medicament from the reservoir. The dose delivery device may include an actuator located at a proximal end of the push rod and configured to advance the push rod in a distal direction through the body. The dose delivery device may include a cannula attached to the distal end of the body through which the medicament from the reservoir is dispensed. The dose delivery system may also include a transponder including a sensor dimensioned to be positioned within the body and configured to detect at least one parameter of a dose of the medicament being dispensed from the reservoir, wherein the sensor may be configured to generate a signal indicative of the parameter. The transponder may include a processor operably coupled to the sensor and configured to process the signal generated by the sensor. The transponder may include an indicator unit operably coupled to the processor and configured to generate an output based on the signal, wherein the output is perceivable by a user. The transponder may also include a battery configured to provide power for at least one of the sensor, the process, and the indicator unit.

Various embodiments of the dose delivery system may include a wireless transmitter that may be configured to communicate with a device located external to the dose delivery device.

### III. BRIEF DESCRIPTION OF THE DRAWINGS

The accompanying drawings illustrate exemplary embodiments of the present disclosure, and together with the description, serve to explain principles of the present disclosure.

FIG. 1A depicts a perspective view of a dose delivery device, in accordance with an embodiment of the present disclosure;

FIG. 1B depicts a partial cross-section of a transponder, in accordance with an embodiment of the present disclosure;

FIG. 2A depicts an embodiment of an exemplary dose delivery device, in accordance with an embodiment of the present disclosure;

FIG. 2B depicts a first configuration of the dose delivery device of FIG. 2A;

FIG. 2C depicts a second configuration of the dose delivery device of FIG. 2A;

FIG. 2D depicts an exemplary embodiment of a transponder, in accordance with an embodiment of the present disclosure;

FIG. 2E depicts another embodiment of an exemplary dose delivery device, in accordance with an embodiment of the present disclosure;

FIG. 3 depicts an exemplary dose delivery device, in accordance with an embodiment of the present disclosure;

FIG. 4A depicts an exemplary dose delivery device, in accordance with an embodiment of the present disclosure;

FIG. 4B depicts an exemplary dose delivery device, in accordance with an embodiment of the present disclosure;

FIG. 5A depicts an exemplary dose delivery device, in accordance with an embodiment of the present disclosure;

FIG. 5B depicts components of the dose delivery device of FIG. 5A;

FIG. 5C depicts components of the dose delivery device of FIG. 5A;

FIG. 6A depicts an exemplary dose delivery device, in accordance with an embodiment of the present disclosure;

FIG. 6B depicts a second configuration of the embodiment of FIG. 6A;

FIG. 6C depicts an exemplary dose delivery device, in accordance with an embodiment of the present disclosure;

FIG. 6D depicts a second configuration of the embodiment of FIG. 6C;

FIG. 6E depicts a third configuration of the embodiment of FIG. 6C;

FIG. 7A depicts an exemplary dose delivery device, in accordance with an embodiment of the present disclosure;

FIG. 7B depicts an exemplary dose delivery device, in accordance with an embodiment of the present disclosure;

FIG. 8A depicts an exploded component of an exemplary dose delivery device, in accordance with an embodiment of the present disclosure;

FIG. 8B depicts exemplary components of the dose delivery device of FIG. 8A;

FIG. 8C depicts an exemplary configuration of the dose delivery device of FIG. 8A;

FIG. 8D depicts a schematic view of the dose delivery device of FIG. 8A;

FIG. 9A depicts an exemplary dose delivery device, in accordance with an embodiment of the present disclosure;

FIG. 9B depicts a second configuration of the dose delivery device of FIG. 9A;

FIG. 10A depicts an exemplary dose delivery device, in accordance with an embodiment of the present disclosure;

FIG. 10B depicts a cross-sectional view of the delivery device of FIG. 10A;

FIG. 11A depicts an exemplary dose delivery device, in accordance with an embodiment of the present disclosure;

FIG. 11B depicts a cross-sectional view of the dose delivery device of FIG. 11A;

FIG. 12A depicts an exemplary dose delivery device, in accordance with an embodiment of the present disclosure; and

FIG. 12B depicts a cross-sectional view an exemplary dose delivery device, in accordance with an embodiment of the present disclosure.

### IV. DETAILED DESCRIPTION OF EMBODIMENTS

Reference will now be made in detail to the embodiments of the present disclosure described below and illustrated in the accompanying drawings. Wherever possible, the same or similar reference numbers will be used throughout the drawings to refer to same or like parts.

While the present disclosure is described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the invention. Accordingly, the disclosure is not to be considered as limited by the foregoing or following descriptions.

Other features and advantages and potential uses of the present disclosure will become apparent to someone skilled in the art from the following description of the disclosure, which refers to the accompanying drawings.

Prior to providing a detailed description of the embodiments disclosed herein, however, the following overview is provided to generally describe the contemplated embodiments. The term “medicament” may be used to refer to a substance used in therapy, a substance that treats, prevents, or alleviates the symptoms of disease, a medicine in a specified formulation, an agent that promotes recovery from injury or ailment, or any other medically active or non-medically active fluid used in the treatment or diagnosis of a patient (e.g., medically inactive saline). The term “distal” may refer to the end of the delivery device that engages the patient. The term “proximal” may refer to the end of the delivery device that is handled by the user. Commercial applications may include those in the medical field, e.g., home care, hospitals, nursing homes, clinics, veterinary uses, and the battlefield or military applications. The delivery device may also be applicable to any non-medical field, e.g. industrial or laboratory uses. Furthermore, elements of the different embodiments may be interchangeably used together in any combination or configuration.

Generally, the embodiments of the present disclosure are directed to a drug delivery dose indicator. The drug delivery dose indicator may comprise a transponder that is associated with a drug delivery device, e.g., a syringe, a pen injector, an autoinjector, or a pump. In some embodiments, the transponder may be positioned within a body of the drug delivery device and may include a sensor to detect a parameter of the delivery device and a processor for determining a characteristic of the dosage dispensation. The transponder may further include an indicator unit configured to emit some type of audio, visual, and/or tactile output to a user to communicate the dose delivery information. The indicator unit may be located on another portion of the delivery device or may be incorporated as part of a single transponder unit. In some embodiments, the transponder may be in communication with an external device (e.g., a smart phone, database, or computer system) configured to organize and/or display information regarding the dose delivery. Other embodiments of the drug dose delivery indicator are also contemplated, as described in the exemplary embodiments below.

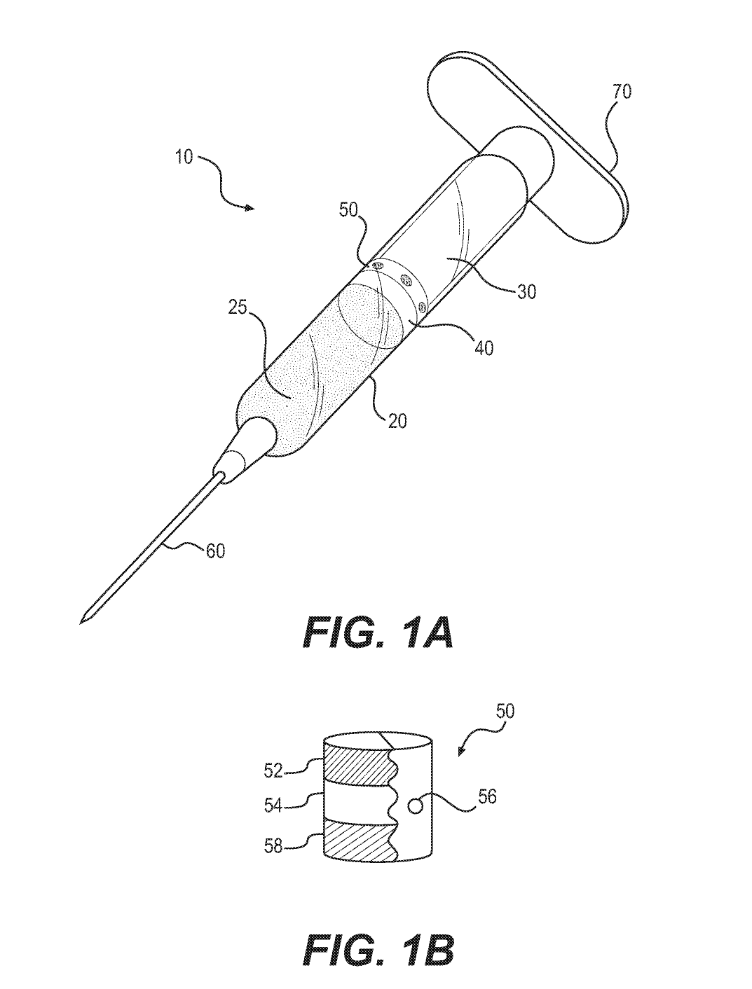

Referring now to the drawings, FIGS. 1A and 1B illustrate a delivery device 10, in accordance with an embodiment of the present disclosure. In the embodiment depicted in FIGS. 1A and 1B, delivery device 10 may include a body 20, a push rod 30, a plunger 40, a cannula 60, and an actuator 70. Body 20 may include a hollow inner volume defining a reservoir 25 configured to contain a medicament. The inner volume may also be configured to contain push rod 30 and plunger 40. Reservoir 25 may be sealed at a proximal end by plunger 40, and the volume of reservoir 25 may vary depending on the location of plunger 40 along the length of body 20. Cannula 60 may define a lumen and may be positioned on a distal end of body 20. A user may activate actuator 70, which advances push rod 30 and plunger 40 to deliver the medicament out of reservoir 25 and through cannula 60. A transponder 50 may be associated with device 10 to detect one or more parameters of the medicament and/or the delivery process.

Transponder 50 may include one or more sensors 52 for detecting at least one parameter of the drug delivery process and a processor 54 in communication with sensor 52. Processor 54 may be configured to interpret a signal generated by sensor 52 and/or to initiate an indication correlating with the sensed parameter. Processor 54 may also be configured to communicate (either wired or wirelessly) data regarding the sensed parameter to a receiver either on or external to the delivery device 10.

Transponder 50 may include an indicator 56 configured to provide an output to a user indicative of the sensed parameter. Indicator 56 may include, e.g., a visual indicator, such as one or more lights, screens, displays, or other suitable devices. Transponder 50 may additionally or alternatively include an audible indicator, such as a speaker configured to generate audible outputs, such as tones, beeps, music, speech, or other audible signals. In some embodiments, transponder 50 may include a structure that generates a tactile output, such as a vibrating mechanism or a mechanism configured to increase or decrease the amount of force required to actuator device 10.

Transponder 50 may also include a lock-out mechanism (not shown) that prevents delivery of a medicament when one or more conditions falls outside of a predetermined acceptable threshold value. For example, the lock-out mechanism of transponder 50 may include projections that engage an inner wall of delivery device 10 to prevent advancement of push rod 30, e.g., when the medicament is not at a suitable temperature for injection, delivery device 10 is not positioned at a suitable angle for delivery, and/or the medicament is being injected at the wrong speed, pressure, acceleration or at the wrong time, for example. In some embodiments, the protrusions may be configured to transition from a low-profile configuration, in which plunger 40 can move past the protrusions, to a lock-out configuration, in which plunger 40 is prevented from passing the protrusions, stopping the delivery of the medicament.

In some embodiments, device 10 may be configured to offset or correct one or more conditions. For example, device 10 may include a mechanism designed to increase the resistance that must be overcome for a user to inject the medicament. Such a mechanism may be activated to slow or correct the acceleration, speed, or pressure at which the medicament is delivered. The mechanism may also be activated to increase the resistance needed for medicament delivery, but not completely prevent medicament delivery, for example, if the medicament is being delivered at the wrong temperature, angle, and/or time.

Transponder 50 may further include a battery 58 that provides power for at least one of sensor 52, processor 54, and indicator 56. Transponder 50 may also include other electronics, such as an antenna, a receiver, any type of memory (e.g., random access memory, flash memory, hard drive), and/or any other suitable components.

Transponder 50 may be disposable or reusable, or may have a combination of reusable and disposable parts, and may be in a number of different positions on device 10 and may have a number of different configurations to determine parameters and/or fault conditions of the injection. For example, transponder 50 may be configured to ensure that delivery device 10 is single use, and may include a lock-out mechanism that prevents device 10 from being actuated a second time. In some embodiments, transponder 50 may be sandwiched between push rod 30 and plunger 40. Transponder 50 may be secured to push rod 30 and/or plunger 40 with an adhesive, a weld, a snap-fit, or other attachment mechanism. Alternatively, transponder 50 may have a loose configuration (e.g. not secured with an attachment mechanism) to facilitate removal and re-use or to allow easy use with an off-the shelf delivery device. In the loose embodiments, transponder 50 may be positioned between push rod 30 and plunger 40, Alternatively, transponder 50 may be positioned within the reservoir or floating in a fluid proximal to plunger 40, e.g., in embodiments in which device 10 does not include a push rod. In another embodiment, transponder 50 may be integrated into device 10. For example, transponder 50 may be integrated into body 20, push rod 30, plunger 40, actuator 70, and/or other suitable components of device 10. Although transponder 50 is shown in FIG. 1B as substantially disk-shaped, transponder 50 may come in any number of other shapes and configurations, e.g., rectangular, pill-shaped, or integrated into device components, with its components being in any suitable arrangement relative to one another.

Sensor 52 may include any suitable structure capable of measuring parameters of the injection process. Sensor 52 may include, for example, one or more of a pressure sensor (e.g. MEMS pressure sensor), a strain gauge, a pressure switch, a timer, a gyroscope, an accelerometer, an optical sensor, a potentiometer, a capacitance sensor, a magnetic sensor, an on/off switch, a displacement sensor, and/or a temperature gauge. Suitable sensors may measure parameters, such as the pressure applied to push rod 30 and/or actuator 70, the length of time that the pressure is applied, the progress of a dose injection, the start and/or stop time of injection, the angle of injection, the position of sensor 52 relative to body 20, the volume of medicament within the reservoir, the flow of fluid within or exiting the reservoir, and/or the temperature of the medicament and/or device 10. Sensor 52 may be configured to generate a signal in accordance with one or more of these measured parameters. The generated signal may be either a binary value or a value proportionate to the measured parameters. Additionally, in some embodiments, relative parameters, rather than actual parameters, may be measured.

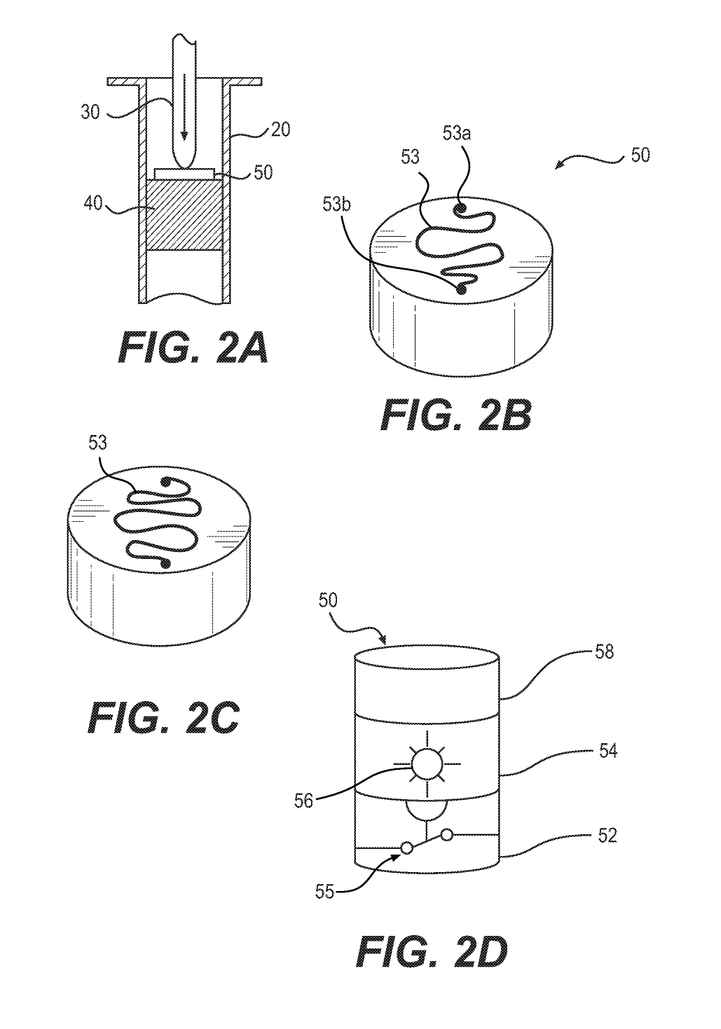

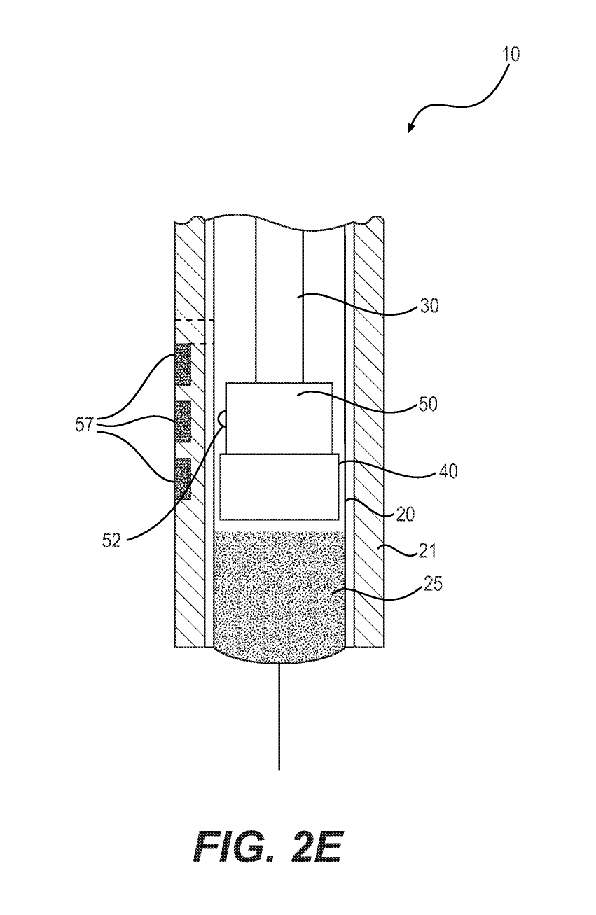

For example, FIGS. 2A-2C depict an embodiment of transponder 50 in which the sensor includes a strain gauge that generates a signal proportionate to the pressure applied to push rod 30 or may detect whether pressure is or is not being applied. The strain gauge may also be configured to detect the static pressure of the medicament. This may be especially evident when the static pressure of the medicament is higher than ambient pressure. The strain gauge may be positioned in any suitable location to determine the pressure applied during the injection process. For example, the strain gauge may be positioned between actuator 70 and push rod 30 or between push rod 30 and plunger 40, as shown in FIG. 2A. The strain gauge may include an electrical conduit 53 extending in a sinusoidal configuration between first and second terminals 53a, 53b. When compressed, the sinusoidal configuration may shorten and become more exaggerated, increasing the resistance, as shown in FIG. 2C. Hence, the voltage measured between first and second terminals 53a, 53b may indicate the pressure applied, which may be outputted by indicator 56 via any suitable visual, audible, and/or tactile mechanism, such as described above.

FIG. 2D depicts an alternative embodiment of transponder 50 in which sensor 52 includes one or more pressure switches 55 configured to detect a number of conditions. Pressure switches 55 may be electrical, chemical, or mechanical structures configured to generate a binary signal based on a threshold pressure applied to push rod 30 and/or actuator 70. Sensor 52 may include a plurality of pressure switches 55 having different thresholds to provide outputs on a number of different conditions. Alternatively, sensor 52 may include one pressure switch 55 to determine a single event, e.g., when a dose is initiated or when an injection pressure is exceeded. For example, a threshold pressure may be achieved when a user activates push rod 30 and/or actuator 70, signaling when a dose is initiated, or when plunger 40 contacts a distal end of housing 20, signaling the end of a dose. Pressure switches 55 may also function as a safety feature detecting, e.g., when a user applies a pressure to push rod 30 that exceeds a suitable threshold pressure, for example, when a dose is delivered too quickly. Pressure switches 55 may initiate any suitable visual, audible, and/or tactile output via processor 54 and/or indicator 56 to indicate a relationship of the detected parameter with one or more threshold pressures. For example, pressure switches 55 may generate an output when the detected pressure meets or is less than or greater than a threshold pressure, and/or when the detected pressure is within a predetermined acceptable range.

As depicted in FIG. 2E, sensor 52 may be configured to interact with a surface of delivery device 10 to detect one or more parameters of the injection process. For example, in an optical embodiment, indicia 57 on device 10 may be detected by an optical sensor 52 to determine the position of transponder 50 relative to body 20. One or more indicia 57 may be embodied in discrete markings, such as a grid or pattern, spaced along device 10. In some embodiments, discrete markers may be evenly spaced or arranged in a gradient, e.g., becoming larger and/or more spaced apart toward a distal end of housing 21, or vice versa. Indicia 57 may alternatively include bar codes or quick response (QR) codes that indicate information to sensor 52. Indicia 57 may be disposed on an inside or outside surface of body 20 or printed on a label disposed on an interior or exterior surface of device 10. As depicted in FIG. 2E, body 20 may be received inside of a housing 21 that surrounds a portion of body 20, and housing 21 may have an inside or outside surface with indicia 57. Body 20 and/or housing 21 may be substantially translucent to make indicia 57 visible to sensor 52. In some embodiments, indicia 57 may be reflective, and sensor 52 may be configured to generate and receive a wavelength of light that is reflected off of indicia 57. In other embodiments, indicia 57 may be configured to generate a wavelength of energy detectable by sensor 52. In yet other embodiments, sensor 57 may be configured to detect a color change of indicia 57. Sensor 52 may include a camera that receives information from indicia 57. Sensor 52 and/or indicia 57 may also be configured to emit energy (e.g., laser or diode) of any wavelength and frequency to determine the relative position of transponder 50. Based on detection of indicia 57, transponder 50 may determine the speed and/or progress of dose delivery according to the relative position data determined by sensor 52. While sensor 52 is shown as separate from push rod 30 and plunger 40, sensor 52 may be incorporated as part of one or more of these delivery device components.

Processor 54 may receive signals generated by sensor 52 and interpret them to assess information regarding the state of injection. In some embodiments, multiple sensors 52 may be incorporated into transponder 50 (or at various locations on delivery device 10), and processor 54 may use one or more of these signals to assess the delivery of a dose (e.g., using data from various sensors to corroborate each other or to assess different parameters). Processor 54 may include, e.g., a timer to determine speed or acceleration and may communicate with a temperature gauge and/or a gyroscope to detect the temperature of the medicament or change in the positioning of device 10 indicative of interruption, etc.

Processor 54 may include or communicate with any number of components that generate an output, e.g., visual, audible, and/or tactile, in response to a signal received from sensor 52 and/or interpreted by processor 54. Processor 54 may embody a single microprocessor or multiple microprocessors capable of monitoring one or more signals from one or more sensors 52. In some embodiments, data from one or more sensors 52 may also be compared with external data or historical data. For example, processor 54 may include a memory, a secondary storage device, a clock, a calendar, a central processing unit, or any other mechanisms for accomplishing a task consistent with the present disclosure. Such configurations may allow a user or healthcare provider to track compliance, efficacy, successful dose delivery, or other relevant information. In some embodiments, processor 54 may communicate (wired or wirelessly) with a receiver of an external processor. Numerous commercially available microprocessors may be configured to perform the functions of processor 54. Various other known circuits may be associated with processor 54, including signal-conditioning circuitry, communication circuitry, and/or other appropriate circuitry.

Processor 54 may also be configured to receive inputs pertaining to the desired treatment. To receive these inputs, processor 54 may be communicatively coupled with an external device, as discussed herein. The inputs may include, but are not limited to, scheduled treatments (e.g. time, date, and dosage amounts), desired temperature of the medicament, or desired speed, pressure, and/or angle of the injection. Processor 54 may utilize the inputs, or the stored data described herein, as thresholds of desired operation of the delivery device 10. For example, a user may need to exert a range of pressures to deliver a drug, and that pressure range may be correlated with a desired threshold range. If a user applies pressure above or below that threshold (plus or minus an acceptable standard deviation), indicator 56 may output a warning to a user. Processor 54 may continually or intermittently compare the signal received from sensor 52 to the predetermined thresholds, and may indicate to the user if a detected parameter falls outside of one or more predetermined thresholds, by generating a fault alert.

The fault alerts may be initiated according to any number of conditions. For example, a fault alert may be initiated if the date and/or time of injection do not correspond with a scheduled treatment. A fault alert may be initiated if the drug is not at a desired temperature range, for example at room or body temperature (e.g., about 2-40° C.). A fault alert may also be initiated when the pressure applied to actuator 70 and/or push rod 30 exceeds a threshold pressure (e.g., about 0.1-10 MPa). This condition may be the result of an inexperienced administrator, a blockage in the cannula, or other device malfunction, and may potentially introduce the drug into the body at an undesirable pressure. A fault alert may further be initiated if the injection is too fast or too slow (e.g. outside range of about 0.005 mm/sec-50 mm/sec), which may be determined by detecting a speed of plunger 40, the flow of medicament, and/or detecting pressure, or if the injection ends without properly delivering the entire dose, for example, if plunger 40, push rod 30, and/or actuator 70 do not reach a final deployed position or final deployment pressure. Another example of a fault signal may be generated when injection does not occur at a desired angle (e.g., 90°±15° or if the angle of injection changes prior to the end of the injection, which may indicate interrupted delivery or that the user is attempting to end delivery before completion.

Processor 54 may notify the user of the detected parameter(s) and/or generate fault alerts with any suitable audible, tactile, and/or visual output or combination thereof. Processor 54 may cause vibration or initiate an audible sound via indicator 56 in response to certain events, such as the end of a dosage or a fault alert being detected. Processor 54 may be operatively connected to indicator 56, which may notify the user of one or more parameters in a number of different manners. As depicted in FIG. 1A, indicator 56 may include one or more light-emitting diodes (LEDs) spaced on an external or otherwise visible portion of transponder 50. LEDs may be arranged in any configuration that may clearly indicate to the user one or more parameter, such as injection start/stop/in-progress, or whether the medicament temperature is ready to start an injection. The LEDs may emit a white or colored light or may change colors to output information. The LEDs may turn on/off to indicate a desirable parameter and then may change to indicate a threshold has been detected. For example, the LEDs may be turned off until the desired temperature has been reached. The LEDs may also be configured to flash at one or more predetermined frequencies, change colors or brightness, or different groups of LEDs may turn on/off to indicate a change in a parameter or that a threshold has been exceeded. The LEDs may also be in a spatial relationship with respect to one another (e.g., stacked in a horizontal or vertical configuration), and the number or orientation of illuminated LEDs in the stack may indicate the dose delivery status, for example, in a manner similar to a battery status bar. In some embodiments, there may be a one-to-one relationship between the number of doses and/or progress of an individual dose delivered and the LEDs illuminated. Even though indicator 56 is described as including one or more LEDs in this exemplary embodiment, the visual component of indicator 56 may additionally or alternatively include electroluminescent lighting, e-ink, and/or any other illumination mechanisms or visual components.

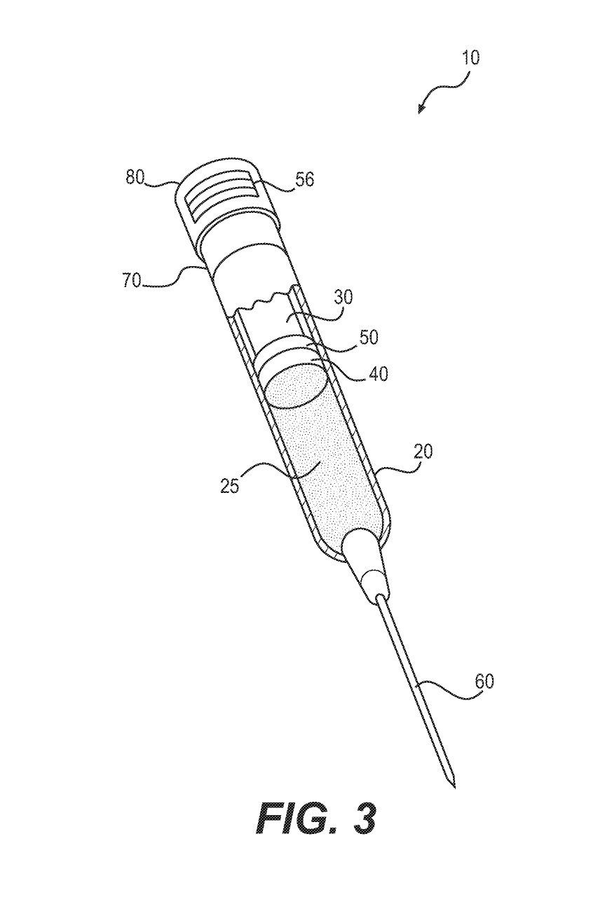

Indicator 56 may be positioned anywhere on delivery device 10, including an external surface of transponder 50, an external surface of body 20, a component that fits onto body 20, or an external device separate from body 20. For example, FIG. 3 depicts delivery device 10, in accordance with another embodiment of the present disclosure, including a cap 80 located at a proximal region with indicator 56 on an external surface. Cap 80 may be permanently or removeably secured to any portion of delivery device 10. For example, transponder 50 and cap 80 may be applied to a standard delivery device or may be integrated as part of a unique delivery device. As depicted in FIG. 3, cap 80 may be positioned on a proximal end of actuator 70. However, it is also contemplated that cap 80 may be positioned on a distal portion of actuator 70, a proximal portion of body 20 or housing 21 (if included), or a distal portion of body 20 or housing 21. Cap 80 may include a receiver and/or transmitter configured to communicate with transponder 50, processor 54, and/or external devices in any wired or wireless manner. Exemplary wireless technologies include, e.g., radiofrequency (RF) and Bluetooth communication.

Cap 80 may include a sensor to generate a signal based on a parameter of the injection or the medicament. The sensor may include, for example, a timer, a gyroscope, an accelerometer, an optical sensor, a potentiometer, a capacitance sensor, a magnetic sensor, an on/off switch, a displacement sensor, and/or a temperature gauge. Suitable sensors of cap 80 may measure parameters, such as the pressure applied to push rod 30 and/or actuator 70, the length of time that the pressure is applied, the progress of a dose injection, the start and/or stop time of injection, the angle of injection, the position of sensor 52 relative to body 20, and/or the temperature of the medicament and/or delivery device 10. Cap 80 may communicate with processor 54 associated with transponder 50 and/or may also include its own processor to process signals generated by the sensor of cap 80, transponder 50, sensor 52, processor 54, and/or external devices. Cap 80 may further include memory (e.g., random access memory, flash memory, hard drive) to store data generated by its own processor or transmitted from transponder 50. The memory and the processor provided in cap 80 may advantageously allow delivery device 10 to process and store data generated by transponder 50 regarding the delivery of the medicament, while obviating the need for processor 54 and reducing the size of transponder 50. Indicator 56 on cap 80 may include a digital screen and/or one or more lights (e.g., LEDs), which may indicate the status of one or more parameters to a user, as further discussed herein. Cap 80 may also include a translucent shell that glows to indicate the status of an injection.

Indicator 56 may indicate any temporal and/or physical aspect of delivery device 10 or medicament within reservoir 25. Indicator 56 may include a plurality of outputs that indicate different parameters and/or fault alerts. Parameters and/or fault alerts may be triggered in response to raw data from sensor 52 or processed data from processor 54. For example, a first output (e.g., a first LED) of indicator 56 may turn on or off when a scheduled treatment is due, and a second output (e.g., a second LED) of indicator 56 may turn on or off when the medicament is in a suitable physical state for injection. Specifically, many types of medication are often stored in a refrigerated environment, but need to be injected at either room or body temperature. When the temperature of the medicament is too cold, the second output of indicator 56 may be turned on or off. If the user attempts to inject the medicament at an undesired temperature, a fault alert may be initiated to indicate to the user that a condition has fallen outside of a predetermined threshold. A similar configuration may be applied to any number of other scheduling or conditions of the medicament, and any number or combination of LEDs or other indicators may be included. Though the above example describes LEDs turning on or off, it is anticipated that they could change color, brightness, blink, or remain steady to output the data to a user. In some embodiments, indicator 56 may include one or more digital and/or analog screens that have colors, graphical, or pictorial indicators of parameters and/or fault alerts. In some embodiments, the screen may output raw data or words, such as “Ready” or “Error.” The screen may also provide a numerical output corresponding with the status of the drug or the progress of the injection. For example, the numerical output may be in form of a percentage or unit of measurement indicating the quantity of a dose remaining to be delivered or the quantity of a dose that has already been delivered. The numerical output may also be in the form of a clock indicating the estimated time remaining for the dosage delivery or an estimated completion time of the dosage delivery.

The visual, audible, and/or tactile output may also indicate a fault condition (user and/or device-related) during the injection process and may have varying levels of output depending on the degree to which a measured parameter falls outside of a threshold. The output may be embodied in varying degrees of light emitted, beeps, words of instruction, clicks, tones, vibrations, and/or pressure feedback. For example, the LEDs may illuminate when the user applies excessive pressure to actuator 70. A degree of brightness may indicate how excessive the pressure is being applied, such that a brighter LED may indicate a higher degree of error. Similarly, the degree of error may be indicated by the frequency of a blinking LED or color. The urgency may be further indicated with the addition of different levels of tactile and/or audible output.

Transponder 50 may also include one or more batteries to power sensor 52, processor 54, indicators 56, and/or actuator 70 or other circuitry. Battery 58 may be any suitable battery known in the art. In some embodiments, battery 58 may be single-use, or in other embodiments, battery 58 may be selectively rechargeable. In such examples, battery 58 may be removed from delivery device 10 and placed into a suitable recharging apparatus until power is fully restored. In such embodiments, battery 58 may be separable from transponder 50, or battery 58 and transponder 50 may be removed together and recharged. In even further embodiments, battery 58 may be configured to be recharged without requiring removal from within delivery device 10, for example, recharged wirelessly via cap 80 and a separate recharging device. In some embodiments, battery 58 may be configured to be recharged by transponder 50.

Body 20, push rod 30, and/or plunger 40 may be any suitable material configured to contain and dispense a medicament. Body 20 may not transmit light, may be at least partially translucent, or may be generally opaque. In embodiments in which body 20 is generally opaque, body 20 may include one or more windows to visualize indicator 56 and/or the medicament within. Alternatively, indicators 56 may be mounted on an external surface of body 20, such as cap 80. Body 20 may be any shape or size configured to contain a medicament, and device 10 may be any suitable drug delivery device, including, for example, any suitable syringe, pen injector, autoinjector, or pump. Body 20, push rod 30, and plunger 40 may embody other pumping mechanisms (e.g., for intravenous therapy). In some embodiments, body 20, push rod 30, and plunger 40 may also be contained in housing 21. However, since delivery device 10 may embody many different drug delivery devices, each of body 20, housing 21, push rod 30, and plunger 40 may be rearranged or omitted from device 10.

Cannula 60 may be any elongate body with a lumen configured to inject the medicament into a body. Cannula 60 may be substantially rigid or flexible and may be formed of any suitable material, including metals, rubbers, and plastics. In some embodiments, cannula 60 may be a needle having a sharp tip to facilitate puncturing of skin. In other embodiments, cannula may be a flexible tube. Cannula 60 may also include any suitable coating desired. For example, the cannula may be coated with anticoagulation, anesthetic, hypoallergenic, anti-inflammatory, lubricant, and/or antibiotic agents. Cannula 60 may have any suitable inner and outer diameter. Further, any suitable number of cannulas may be included in delivery device 10. Any number of medicament delivery cannula 60 may be used, for example, one, two, three, or more cannula. Each cannula 60 may deliver one or more types of fluid, and may include one or more channels for delivering fluid. If more than one cannula and/or fluid type is used, then multiple transponders 50 and/or sensors 52 may be used to provide a user with feedback for each.

Actuator 70 may be any device configured to translate push rod 40 in order to expel medicament from reservoir 25. As depicted in FIG. 1A, actuator 70 may be a thumb rest integrated into the proximal end of pusher rod 40. Depressing actuator 70 may advance push rod 40 to inject the medicament. Actuator 70 may include a ratcheting mechanism to provide controlled dose delivery. In some embodiments, actuator 70 may include stored energy provided by, e.g., springs, compressed gas, an electrical motor, or a rotary driver. In some embodiments, actuator 70 may include a leaf spring that may be biased into a coil configuration to advance push rod 30. In operation, the leaf spring may be lengthened into a generally elongate configuration. A restraining feature (e.g. pin, clip, or the like) may be configured to maintain the leaf spring in the elongate configuration. Once the restraining feature is removed, the leaf spring may return to the coil configuration to advance push rod 30. Other embodiments of actuator 70 may include a hydraulic mechanism, which may advance plunger 40 with fluid pressure. This hydraulic mechanism may obviate the need for various components, such as push rod 30.

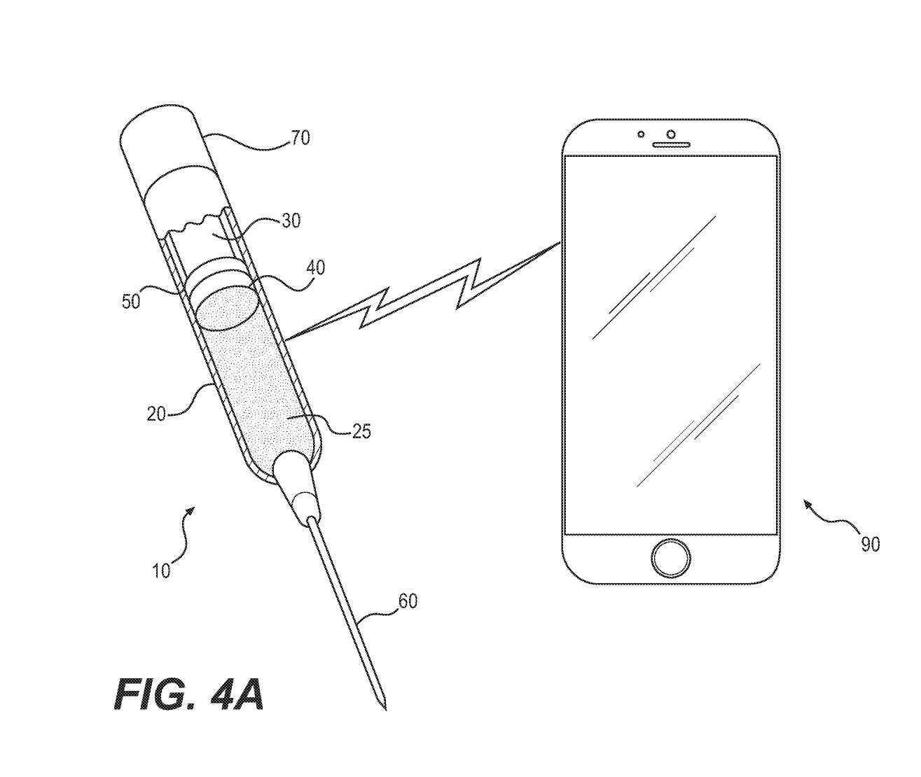

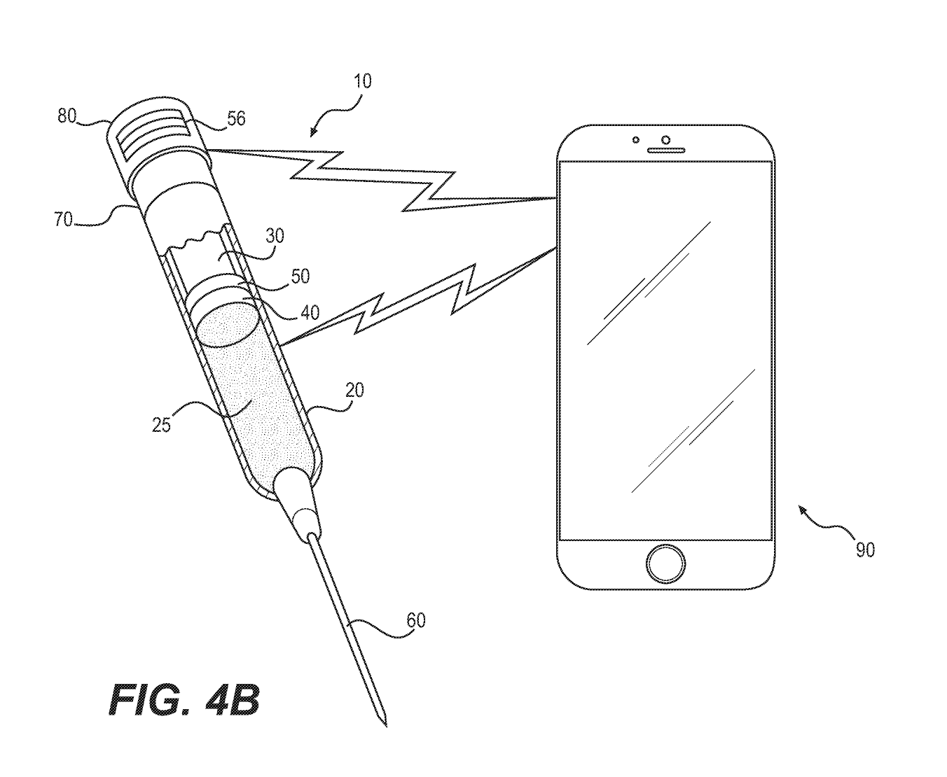

FIGS. 4A and 4B depict delivery device 10 in wireless communication with an external device 90 via one or more transmitters and/or a receivers, in accordance with additional embodiments of the present disclosure. Transponder 50, cap 80, and/or external device 90 may communicate with each other regarding any parameter and/or fault condition concerning, e.g., an injection of medicament or a treatment schedule. One or more of external device 90, transponder 50, and/or cap 80 may include a transmitter, a receiver, and/or a processor for facilitating communication. As shown in FIG. 4A, transponder 50 of delivery device 10 may communicate directly with external device 90, either transmitting, or both transmitting and receiving, data between the components. As shown in the embodiment of FIG. 4B, delivery device 10 may include both transponder 50 and cap 80. In such an embodiment, transponder 50 and cap 80 may both communicate with external device 90. Transponder 50 and cap 80 may both transmit and/or receive data with external device 90, only one of transponder 50 and cap 80 may transmit and/or receive data with external device 90, or one of transponder 50 and cap 80 may transmit and/or receive data with the other component on delivery device 10 while only one of the components transmits and/or receives data with external device 90. For example, in embodiments in which transponder 50 and cap 80 both have sensors to detect one or more parameters, each of transponder 50 and cap 80 may relay to external device 90 data about what each sensor detected. In some embodiments, only one of cap 80 and transponder 50 may communicate with external device 90 while cap 80 and transponder 50 communicate with each other. For example, transponder 50 may communicate with cap 80, and cap 80 may in turn communicate with external device 90, or vice versa. Even though wireless communication is depicted in FIGS. 4A and 4B, transponder 50, cap 80, and/or external device 90 may, additionally or alternatively, communicate with each other via hard-wired connections, or via a combination of hard-wired and wireless connections with one another.

Further, to facilitate communication, raw and/or processed data generated by the sensors of transponder 50, cap 80, and/or external device 90 may be processed and/or stored in any one or more of transponder 50, cap 80, and/or external device 90. This feature may reduce the size of transponder 50 by obviating the need for certain components housed in transponder 50 (e.g. processor 54 and/or memory). For example, raw data generated by sensor 52 may be transmitted to cap 80 to be processed by the processor of cap 80 along with raw data from the sensor of cap 80. In one embodiment, to ensure that an entire dose is delivered prior to the delivery device 10 being removed from the patient, sensor 52 may generate raw data corresponding to the pressure applied by push rod 30, while a gyroscope housed in cap 80 may generate raw data corresponding to the angular pitch of delivery device 10. The processor of cap 80 may process the sets of raw data generated by transponder 50 and cap 80, and initiate an output if delivery device 10 was rotated prior to the dose being completely delivered.

External device 90 may include one or more smart phones, smart watches, wearables, patient monitors, tablets, computers, medical databases, or any other technology, and may receive, display, and/or store the parameter and/or fault condition in any known manner. For example, external device 90 may store the parameter and/or fault condition received from transponder 50 and/or cap 80 on an internal or external hard drive or upload it onto a server. The information regarding the parameters may be displayed in a calendar of scheduled treatments, may link to specific directions pertaining to the injection of the medicament, may be included with emergency contact information, or may be used by itself or in combination with other information to update a treatment regimen. In some embodiments, external device 90 may be used as an external indicator, instead of, or in addition to, indicator 56. External device 90 may notify the user of parameters and/or fault conditions with any visual, audible, and/or tactile output. External device 90 may also be configured to notify third parties (e.g., healthcare professionals) of the parameters and/or fault conditions. External device 90 may be further configured to allow the user to input data to be communicated to transponder 50, cap 80 (if included), and/or processor 54 (which may be located on one or both of transponder 50 and cap 80), concerning the desired treatment. For example, external device 90 may be used to input or change threshold parameters, as further discussed herein. Exemplary applicable wireless technologies may include, e.g., RF and Bluetooth communication.

In some embodiments, transponder 50, processor 54, cap 80, and/or external device 90 may alter the threshold parameters or the treatment schedule based on the information detected by transponder 50 and/or cap 80. This alteration may be automatic or may be done manually. For example, parameters of actual medicament injections (e.g., those detected by transponder 50) may be compared to a patient treatment schedule (e.g., stored on external device 90) to determine whether the treatment schedule should be modified. Specifically, if a patient has skipped one or scheduled medicament dosage deliveries, or delivered an incomplete dose of medicament, subsequent treatments may be increased or decreased, to accommodate this change. The modification may be performed automatically (e.g., through a processor of external device 90), and/or manually (e.g., by a healthcare professional in communication with external device 90). The modified treatment schedule may then be communicated (e.g., from external device 90 to transponder 50 and/or cap 80), to be implemented by delivery device 10. Thresholds of individual scheduled treatments may then be increased, reduced, added, and/or cancelled in order to promote proper administration of medicament and/or to monitor subsequent treatments, as discussed herein.

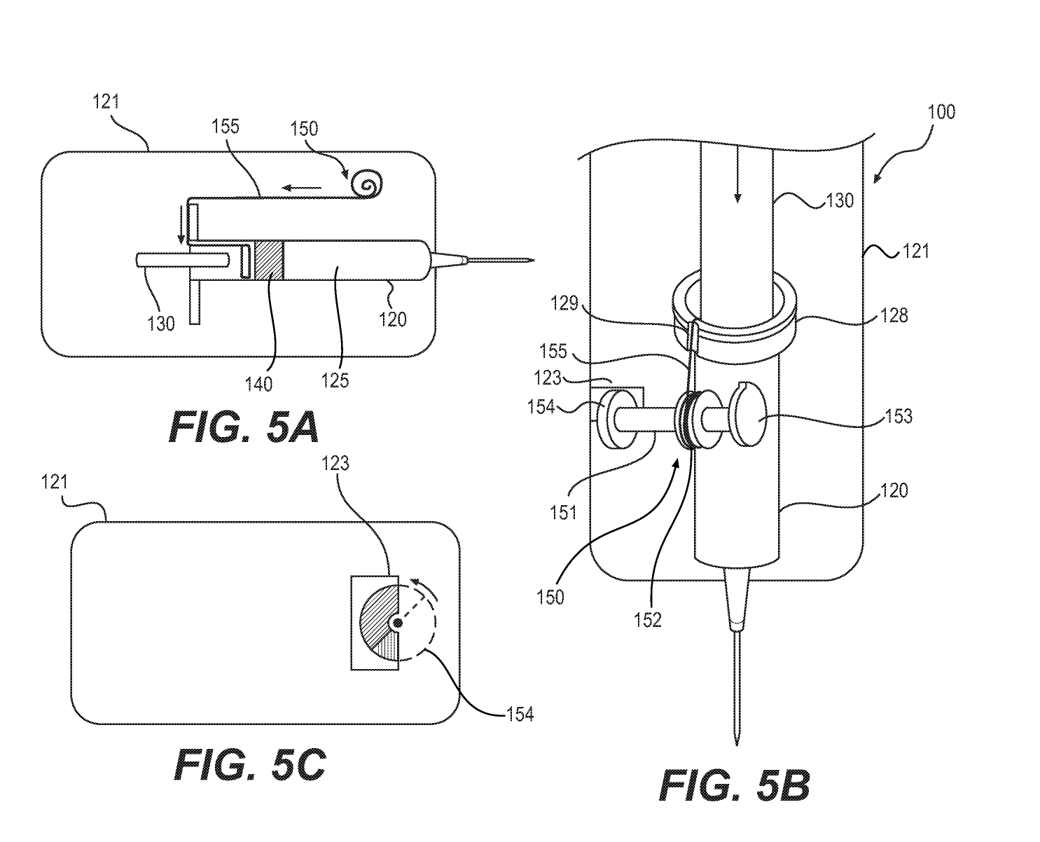

FIGS. 5A-5C disclose another embodiment of a delivery device 100 comprising a rotary mechanism 150 configured to measure the dispensing of medicament. Delivery device 100 may include a body 120 that contains a push rod 130, a plunger 140, and a reservoir 125. Body 120 may also include a cable guide 128 on a proximal region, defining a flange with a longitudinal slot 129. Rotary mechanism 150 may include an axle 151 that may support a spindle 152, a clicker 153, and an indicator 154. Clicker 153 and/or indicator 154 may be in the form of a full wheel or a radial segment extending around axle 151. Delivery device 100 may further include a cable 155 having a first end secured to spindle 152, an intermediate portion that extends over cable guide 128 though longitudinal slot 129, and a second end secured to plunger 140. The first and second ends of cable 155 may be secured with an adhesive, a cleat, a clip, a clamp, a crimp, or other suitable securing mechanisms. Delivery device 100 may be contained within a housing 121 defining a window 123 aligned with indicator 154.

In operation, push rod 130 may advance plunger 140 to deliver medicament from reservoir 125. As plunger 140 is advanced, cable 155 may be pulled from rotating spindle 152 to translate rotational motion to axle 151, clicker 153, and indicator 154. Axle 151 may create a resistance to maintain tension in cable 155 while being pulled. Axle 151 may be rate controlled, controlling the speed of injection. The amount of strain on axle 151 and/or cable 155 may be measured by a sensor to determine parameters of the injection process. Axle 151 may also include a ratcheting mechanism to provide controlled delivery. Clicker 153 may be configured to emit vibrations and/or audible output indicative of one or more parameters. Indicator 154 may include indicia providing dosage information and may be visible through window 123, depicted in FIG. 5C. The dosage information may include the progress of the current dosage, the quantity of the remaining doses in reservoir 125, and/or any other parameter and/or fault alert discussed in this application.

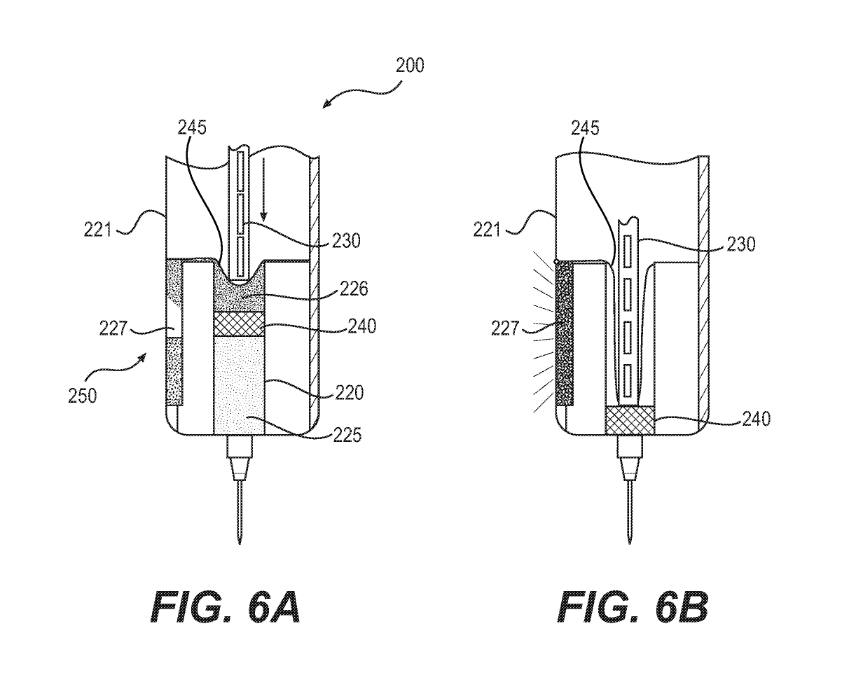

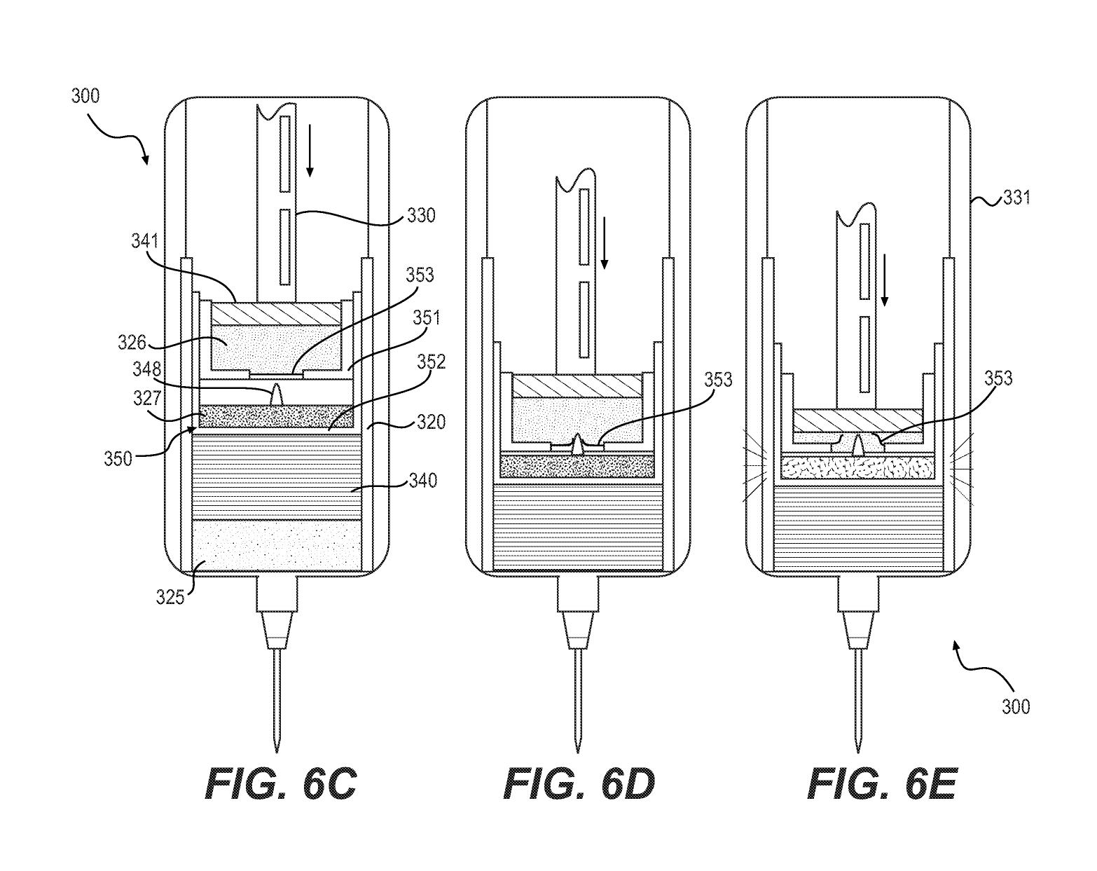

FIGS. 6A-6B disclose another embodiment of a delivery device 200 having a chemiluminescent indicator 250 with exterior mixing to indicate to a user information regarding the progress of an injection. Delivery device 200 may include a push rod 230 and a body 220. Body 220 may define a first reservoir 225 distal of a plunger 240 for containing a medicament, and a second reservoir 226 proximal of plunger 240 for containing a first reagent. Plunger 240 may seal a proximal end of first reservoir 225 and a distal end of second reservoir 226 so that the contents of the first and second reservoirs 225, 226 do not mix. Second reservoir 226 may also be initially sealed at the proximal end by a membrane 245. A housing 221 may be located exterior to body 220, either completely encasing body 220 or extending along an exterior portion of body 220, and may define a third reservoir 227 configured to contain a second reagent. In one embodiment, housing 221 may include a window in-line with third reservoir 227. In another embodiment, housing 221 may be completely translucent.

Second and third reservoirs 226, 227 may contain one or more reagents that undergo a transformation (e.g. change colors) when mixed. In some embodiments, second reservoir 226 may contain a chemiluminescent agent, while third reservoir 227 may contain an oxidizing agent, or vice versa. The chemiluminescent agent may include, e.g., luminol, and the oxidizing agent may include, e.g., iron and/or copper filings. In other embodiments, the reagents may be dyes or chemicals that may change color when mixed. In yet other embodiments, third reservoir 227 may initially be empty, and may be filled by fluid transferred from second reservoir 226.

In operation, chemiluminescent indicator 250 may be configured to indicate the advancement of push rod 230. Specifically, push rod 230 may be configured to displace membrane 245 to disrupt the seal at the proximal end of second reservoir 226. Advancement of push rod 230 and displacement of membrane 245 may further force the chemiluminescent agent out of second reservoir 226 into third reservoir 227 to mix with the oxidizing agent. The mixture may generate a chemiluminescent glow visible to user. Membrane 245 may also be configured to function as a pump to propel chemiluminescent agent into third reservoir 227 with advancement of push rod 230, to ensure mixing. The quantity of the chemiluminescent mixture may indicate the advancement of push rod 230, and third reservoir 227 may be sized to be substantially full of chemiluminescent mixture when substantially all the medicament is delivered from first reservoir 225 and membrane 245 is fully deformed, as depicted in FIG. 6B. The chemiluminescent glow may be readily visible to a user, and the strength of glow may also indicate the progress of push rod 230 and thus dose delivery.

FIGS. 6C-6E discloses another embodiment of a delivery device 300 having a chemiluminescent indicator 350 with interior mixing. Delivery device 300 may include a body 320, a push rod 330, a first reservoir 325, a first plunger 340, and a second plunger 341. Chemiluminescent indicator 350 may include a first partition 351 and a second partition 352. First partition 351 may have a semi-cylindrical shape, and may be located surrounding distal portion of push rod 330. First partition 351 may define a second reservoir 326 configured to contain a chemiluminescent agent, and may include a central membrane 353 on a distal surface. Second partition 352 may also have a semi-cylindrical shape and define a third reservoir 327 configured to contain an oxidizing agent. Second partition 352 may be releasably fixed within first partition 351 and include a tine 348, or other sharpened member, extending in the direction of membrane 353. Second partition 352 may be releasably fixed within first partition 351 with a releasable mechanism, such as a frangible member, an interference fit, a hydraulic mechanism, or the like.

Initial advancement of push rod 330 may be used to advance first partition 351, second partition 352, and first plunger 340 together to dispense medicament in first reservoir 325. After sufficient force is applied to dispense the dose of medicament, releasable mechanism may release first partition 351 relative to second partition 352, allowing relative movement of first partition 351 in a distal direction towards second partition 352. First partition 351 may then be advanced such that membrane 353 may contact tine 348 causing membrane 353 to rupture. Chemiluminescent agent from first reservoir 346 may then mix with oxidizing agent from second reservoir 327, generating a chemiluminescent glow. Body 320 may be at least partially translucent to allow user to visualize the chemiluminescent glow or may include a window (not shown) through which it is viewed.

Chemiluminescent indicator 350 may indicate a number of conditions. In some embodiments, the releasable mechanism may be configured to allow relative movement between first partition 351 and second partition 352 when plunger 340 reaches the distal surface of housing 320. In this embodiment, the chemiluminescent glow may indicate completion of dose delivery. In another embodiment, chemiluminescent glow may indicate the initiation of dispensing. Releasable mechanism may also be configured to initiate the chemiluminescent glow if excessive pressure is applied to push rod 330.

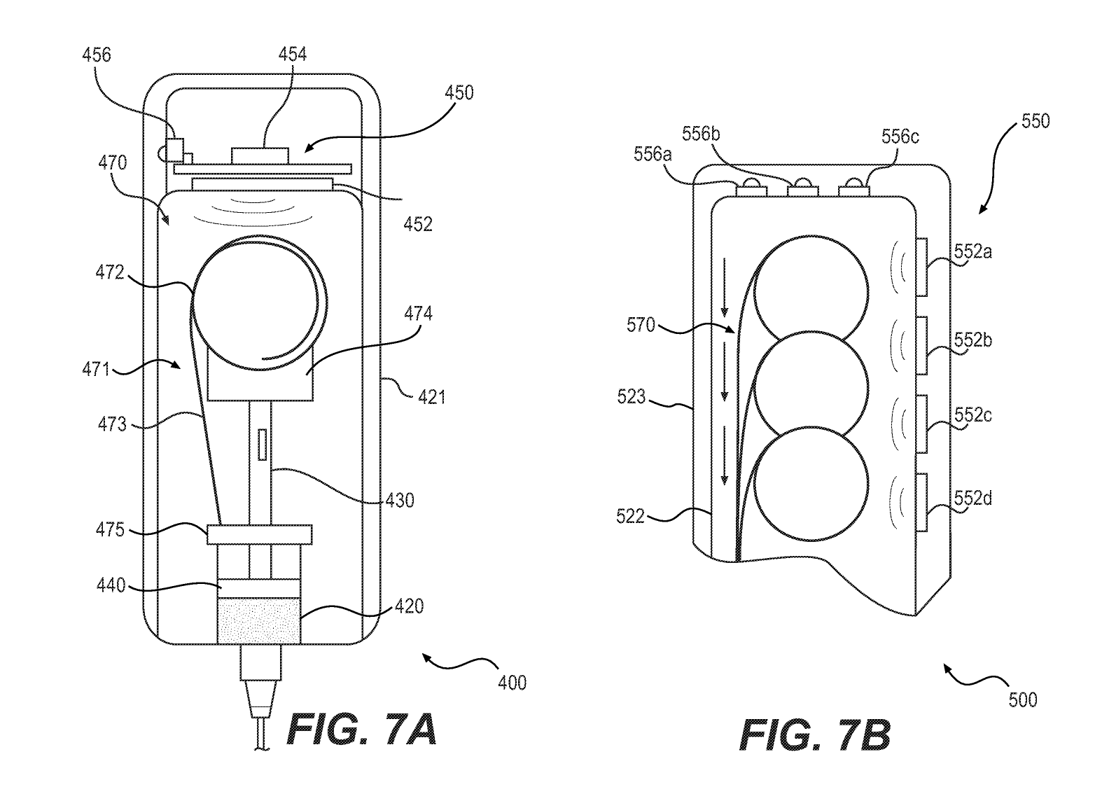

FIG. 7A discloses another embodiment of a delivery device 400 having a metal detector indicator system 450. Delivery device 400 may comprise a body 420, a push rod 430, a generator 452, and an actuator 470. Actuator 470 may further include a leaf spring 471 having a helical portion 472 connected to a first member 474 and an elongate portion 473 connected to a second member 475. Leaf spring 471 may be biased into a helical configuration, such that when unrestrained, leaf spring 471 may push first member 474 to advance push rod 430. Prior to actuation, a restraining feature (e.g. a pin, a clip, a hook, or the like) may be configured to maintain leaf spring 471 in the elongate configuration. Leaf spring 471 may include a magnetic material that can be detected by generator 452.

Metal detector system 450 may include any structure to detect the location of the metallic portion of actuator 470. Metal detector system 450 may include one or more generators 452, a processor 454, and an indicator 456 positioned on a proximal end of body 420 within housing 421. Generator 452 may be configured to transmit a first magnetic field toward actuator 470 to produce a second magnetic field in the metallic portion of actuator 470. Generator 452 may then detect the second magnetic field, which may then be used to determine the relative location of actuator 470. Generator 452 may also detect any change in the first magnetic field to determine the relative location of actuator 470. As in the other embodiments, processor 454 may determine any number of characteristics of the delivery of a medicament based on the data detected by generator 452, and indicator 456 may output information to the user in any number of suitable manners.

FIG. 7B discloses another embodiment of a delivery device 500 having a metal detector system 550. Metal detector system 550 may include one or more generators 552 connected to one or more indicators 556. Generators 552a-d may have a similar configuration as in the embodiment of FIG. 7A, but may be positioned along a housing 521. Generators 552 may be on an inner or outer surface of a housing 521. As the metal portion of an actuator 570 passes generators 552a-d, a corresponding indicator 556a-c may generate an output signaling the progress of the injection process. Proximal generator 552a may be adjacent the starting position of actuator 570, and distal generator 552d may be positioned adjacent a completion position of actuator 570. Any number of intermediate generators 552 may be positioned between proximal generator 552a and distal generator 552d. For example, generators 552 may be positioned along the side of housing 521 based on the volume of individual doses to indicate the quantity delivered in a multi-dose device or may be used to indicate the progression of a single dose.

Delivery device 500 may have any suitable number of indicators 556 located on device 500 (e.g., along or within the body or housing 521). In one embodiment, generators 552 may be arranged in a single circuit with one or more indicators 556. This embodiment may allow the output of indicators 556 to accumulate as additional output from generators 552 is produced. In another embodiment, each generator 552 may be on a separate circuit to indicate position of actuator 570. In this embodiment, there may be a one-to-one relationship between generators 552 and indicators 556, such that as actuator 570 passes a generator 552, a corresponding indicator 556 may generate an output. Indicators 556 may cause an outer shell 523 to glow depending on the progression of actuator 570, e.g., via use of one or more indicators turning on/off, brightening, blinking, or changing colors.

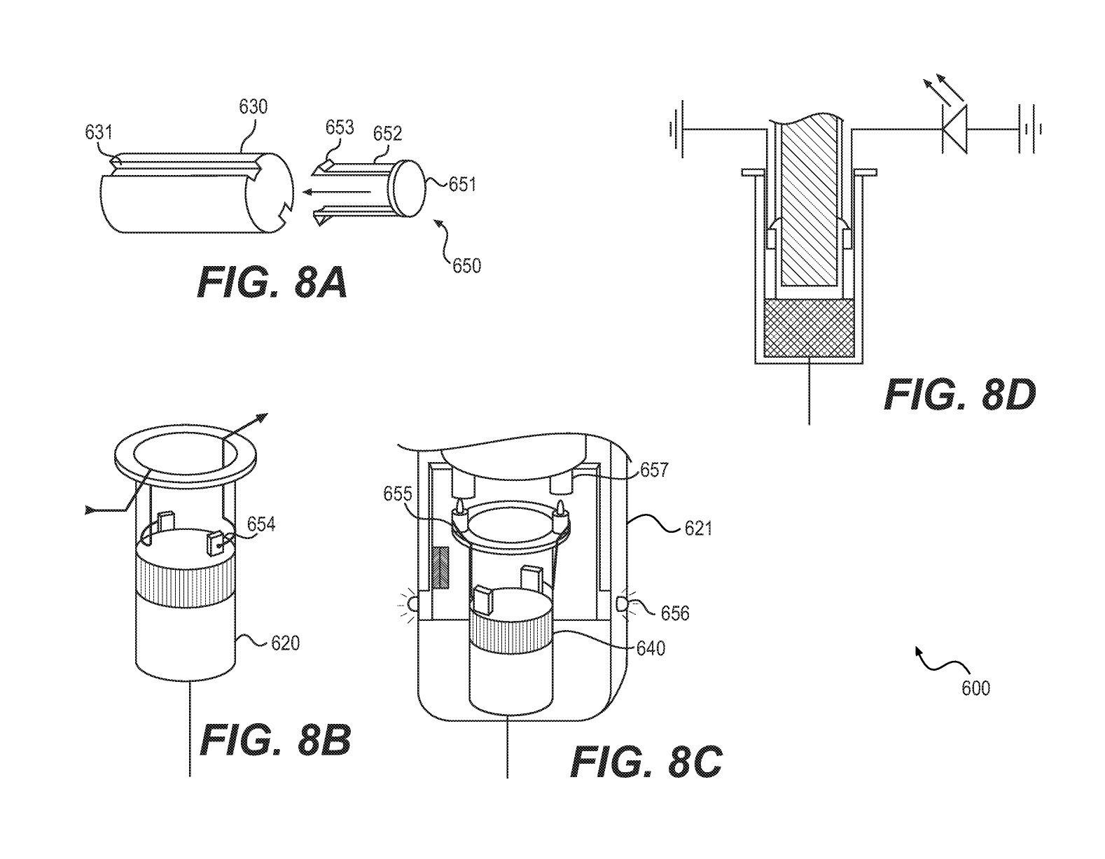

FIGS. 8A-8D disclose another embodiment of delivery device 600 having a contact tip 650. As shown in FIG. 8A, delivery device 600 may include a push rod 630 having one or more longitudinal grooves 631. Contact tip 650 may be received on a distal end of push rod 630. Contact tip 650 may include a generally circular base 651 received distally of push rod 630, one or more legs 652 received in longitudinal grooves 631, and at least one protrusion 653 at the proximal end of each leg 652. Contact tip 650 may include a conductive material. As shown in FIG. 8B, one or more contact points 654 may be positioned on an inside surface of a body 620 proximal of a plunger 640. In one embodiment, body 620 may include two contact points 654, and contact tip 650 may have two legs 652 configured to interact with contact points 654.

As shown in FIGS. 8C-8D, contact points 654 may be a component of an electrical circuit including contact tip 650 and indicators 656. Indicators 656 may include one or more LEDs that illuminate or otherwise indicate when a signal is generated across the circuit and may be positioned on an outer surface of body 620 or a housing 621 visible to a user. This circuit may be incomplete until contact tip 650 is advanced past contact points 654. The advancement of push rod 630 may cause base 651, legs 652, and/or protrusions 653 to engage contact points 654 to complete the circuit, as contact tip 650 advances past contact points 654. In one embodiment depicted in FIG. 8C, body 620 may be releaseably secured within housing 621. In this embodiment, the circuit may include body connectors 655 and housing connectors 657 that are releasably connectable in order to place indicators 656 in connection with contact points 654.

Contact tip 650 may have a variety of different configurations providing different portions of contact tip 650 to contact points 654 as push rod 630 is advanced. In one embodiment, contact tip 650 may be sized such that contact tip 650 only engages contact points 654 at protrusions 653. This configuration may cause the indicator 656 to illuminate only when the dose is complete. In another embodiment, contact tip 650 may be sized such that contact points 654 engage base 651 and protrusions 653. In this embodiment, indicators 656 may illuminate immediately prior to the injection of the dose, and when the dose is complete. In yet another embodiment, contact tip 650 may engage contact points 654 along the length of base 651 and legs 652. The circuit may be configured to increase the signal generated as the contact tip 650 is advanced along contact points 654. This embodiment may allow the user to determine the progression of the injection based on the intensity of indicators 656.

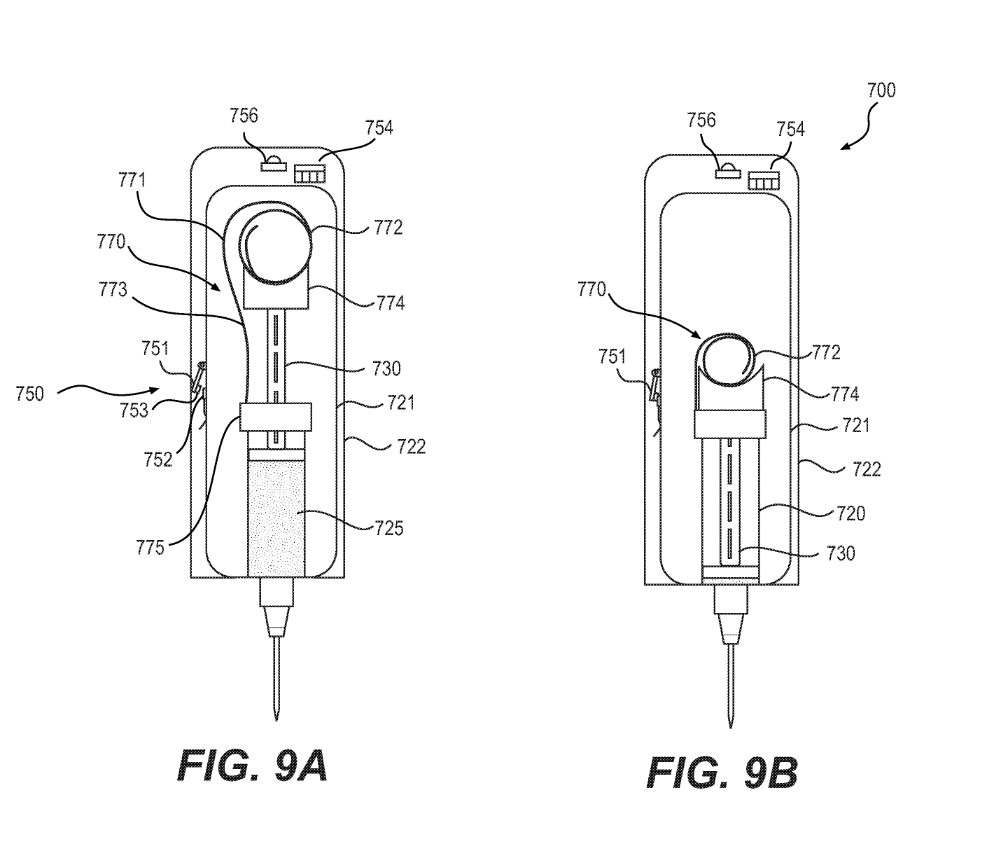

FIGS. 9A-9B disclose another embodiment of delivery device 700 having at least one magnetic sensor for detecting the location of the magnet as it passes. In some embodiments, as depicted in FIGS. 9A-9B, the magnetic sensor may be in the form of a magnetic switch 750. Delivery device 700 may include a body 720, a housing 721, a shell 722, a push rod 730, a magnetic switch 750, and an actuator 770. Actuator 770 may further include a metallic member 771 having a helical portion 772 connected to a first member 774 and an elongate portion 773 connected to a second member 775. As shown in FIG. 9A, metallic member 771 may be biased into the helical configuration, such that when unrestrained, metallic member 771 may push first member 774 to advance push rod 730. Prior to actuation, a restraining feature (e.g. pin, clip, hook, or the like) may be configured to maintain metallic member 771 in the elongate configuration. Metallic member 771 may include a magnetic material that interacts with one or more switches 750.

Switches 750 may be positioned anywhere along the length of housing 721. As depicted in FIGS. 9A-9B, switch 750 may be positioned substantially aligned with the fully advanced position of actuator 770. Switch 750 may include a pivoting arm 751 with a magnetic electrode 752 on one end. A second electrode 753 may be on an exterior surface of housing 721. First electrode 752 and second electrode 753 may be wired to form a switch for a circuit with a processor 754, and an indicator 756 contained within housing 721 or shell 722. As further depicted in FIG. 9A, switch 750 may be open when actuator 770 is positioned proximal of switch 750 (e.g., when actuator is in a pre-deployed or semi-deployed state). As further shown in FIG. 9B, switch 750 may close when actuator 770 becomes substantially aligned with switch 750 (e.g., in a fully deployed state) due to the magnetic attraction between metallic member 771 and first electrode 752. Switch 750 may be biased in the open configuration. When the circuit is closed, the indicator 756 may be powered to illuminate or vice versa. A plurality of switches 750 may be positioned along the length of housing 721 to further indicate the progression of actuator 770, to output information regarding the progress of dose delivery. In one embodiment, each switch 750 may be connected to one of a plurality of indicators 756 to indicate the delivery doses dependent on the positioning of actuator 770. For example, switches 750 may be configured, such that only a single switch 750 closes depending on the location of actuator 770, to illuminate a single indicator 756 indicating the location of push rod 730. The plurality of switches 750 may be configured in a single circuit or multiple circuits, as discussed in the embodiments above. In another embodiment, indicator 756 may be steady when push rod 730 is at a proximal-most and a distal-most position, and indicator 756 may blink as the actuator 770 advances past switches 750.

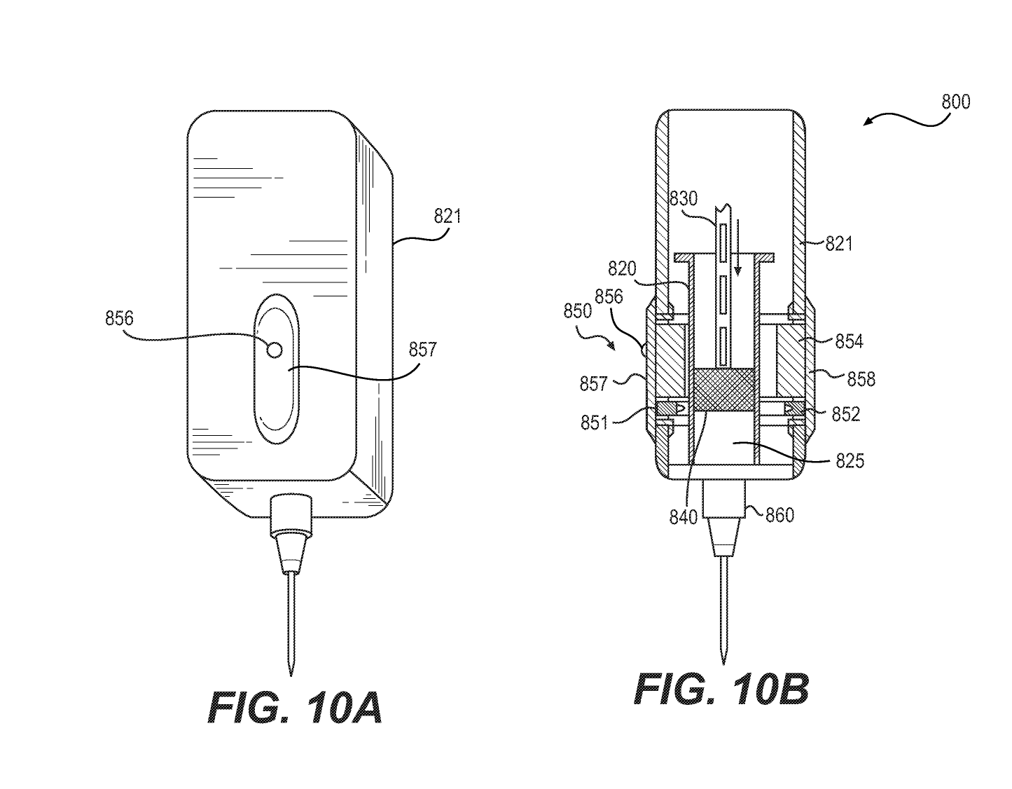

FIGS. 10A-10B disclose another embodiment of delivery device 800 with an optical indicator system 850. Optical indicator system 850 may include at least one emitter 851, at least one detector 852, at least one processor 854, and at least one indicator 856. In the embodiment of FIGS. 10A-10B, indicator 856 is included in a set of first and second caps 857, 858, which may be removably or permanently coupled to an external surface of housing 821. Emitter 851 may be configured to generate and emit a wavelength of energy and direct that wavelength along a linear pathway toward detector 852. Detector 852 may receive the wavelength of energy and generate a signal upon receipt.

As in the previous embodiments, push rod 830 may advance plunger 840 to push medicament out of cannula 860. In this embodiment, optical indicator 850 may be configured to determine the location of plunger 840 based on an interruption of the wavelength of energy from emitter 851. Optical indicator 850 may have any number of emitters 851 and detectors 852, positioned at locations along the longitudinal axis of body 820. Emitters 851 and detectors 852 may be spaced along the longitudinal axis to indicate discrete doses being delivered or an amount of a dose that has been delivered. Once a wavelength of energy from one of the emitters 851 is interrupted and detector 852 fails to receive the energy, one of the indicators 856 may signal to the user that a dose was delivered or that a certain amount of a dose has been delivered. One of the emitters 851 may be positioned immediately proximal of plunger 840 when it is in the distal-most position within body 820 to indicate that reservoir 825 is empty. Processor 854 may determine the speed of plunger 840 based on the change in position detected by detectors 852. Optical indicator system 850 may emit energy of any wavelength and frequency sufficient to determine the location of plunger 840. Optical indicator system 850 may include, for example, one or more of a laser, a diode, or any other type of known technologies.

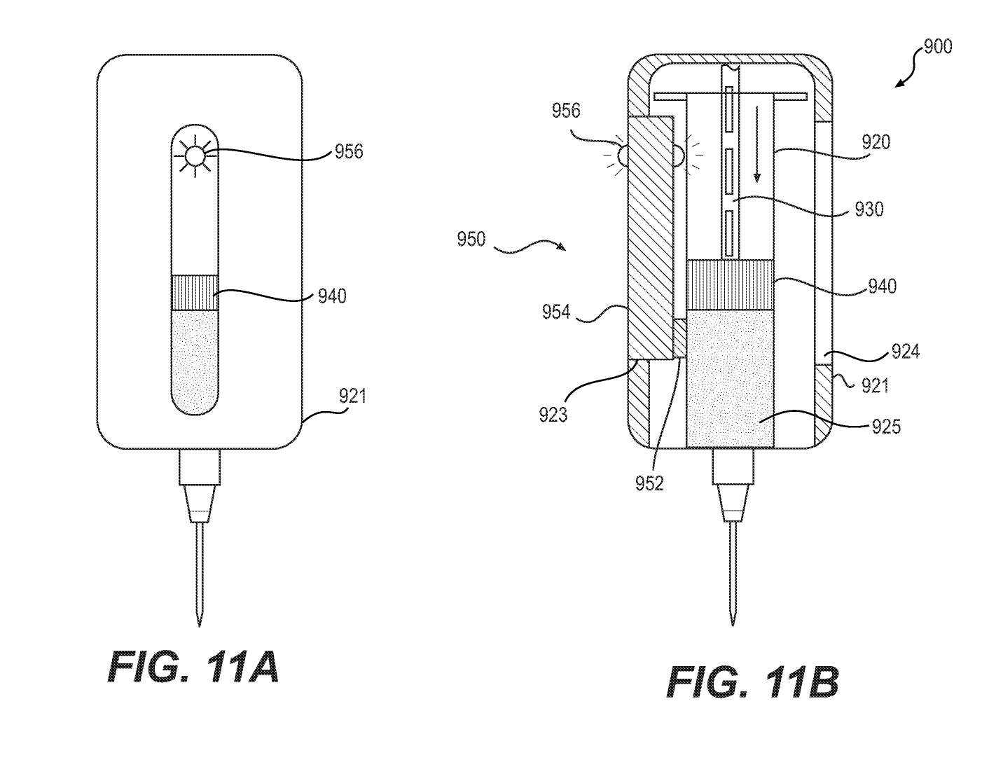

FIGS. 11A-11B disclose another embodiment of a delivery device 900 having a capacitance sensing system 950. Capacitance sensing system 950 may include a sensor 952, a processor 954, and an indicator 956. Sensor 952 may include any material capable of creating a capacitance to detect the medicament in reservoir 925. The material may include, e.g., copper, indium tin oxide (ITO), and/or printed ink. Sensor 952 may create a small power across its surface, which is altered by the presence of the medicament in reservoir 925. Sensor 952 may also include a grid of electrodes that is configured to determine the level of medicament and output a proportional signal. Sensor 952 may be responsive to temperature, moisture, electrical conductivity, or any other property characteristic of the medicament. Processor 954 may be configured to process the signal and generate an output via one or more indicators 956. For example, delivery device 900 may include a housing 921 having a first and second window 923, 924. Processor 954 may be permanently or removeably positioned within first window 923, while second window 924 may be substantially translucent. Therefore processor 954 may be provided with two indicators 956 on first and second sides visible to the user, as depicted in FIG. 11A. Indicator 956 may, additionally or alternatively, be positioned on an exterior of delivery device 900 and sensor 952 may be hidden. Housing 921 may also be entirely substantially translucent to allow for visualization of indicator 956.

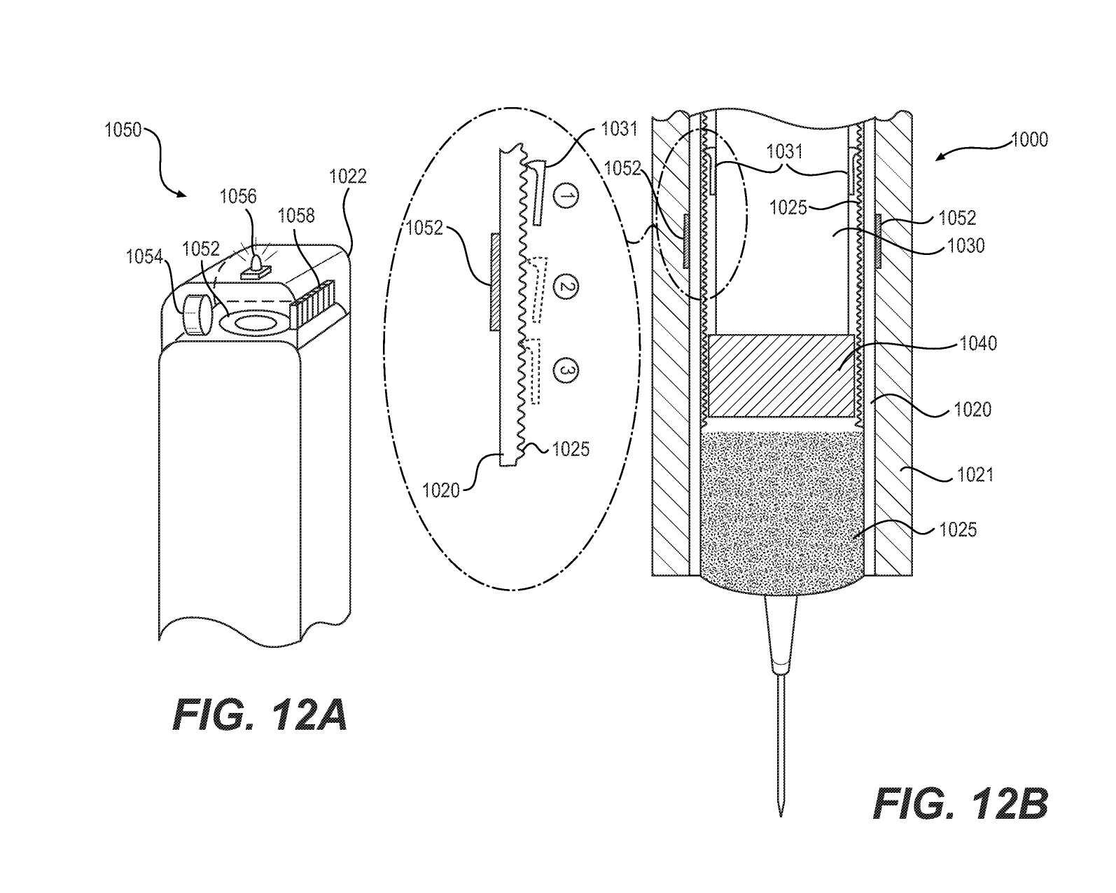

FIGS. 12A-12B disclose another embodiment of delivery device 1000 having a piezoelectric sensor system 1050. Delivery device 1000 may include a number of different mechanisms configured to generate clicks and/or vibrations indicative of the delivery of the medicament. In one embodiment, push rod 1030 may include a tooth 1031 biased into engagement with a sawtooth region 1025 located on an inner surface of either body 1020 or housing 1021. Alternatively, rather than being attached to push rod 1030, tooth 1031 may be attached to or associated with plunger 1040, or an actuator (not shown), or on an inner surface of body 1020 or housing 1021. Consequently, sawtooth region 1025 may be positioned in a number of locations to correspond with tooth 1031, for instance, when tooth 1031 is located on housing 1021 or body 1020, sawtooth region 1025 may be located on push rod 1030, plunger 1040, or body 1020. As push rod 1030 is advanced with respect to the inner surface, tooth 1031 may be dragged along sawtooth region 1025, deflecting away from the inner surface and then re-contacting the inner surface to generate a series of clicks and/or vibrations.

Piezoelectric sensor system 1050 may include a sensor 1052, a processor 1054, and an indicator 1056 that may be positioned within a shell 1022. Sensor 1052 may include any mechanism capable of generating a signal based on the detection of any clicks and/or vibrations generated by delivery device 1000. These clicks or vibrations generated by tooth 1031 and sawtooth region 1025 may be indicative of medicament delivery; for example, the starting or stopping of clicks may indicate the start or stop of drug delivery, the speed of clicks or volume of clicks may indicate speed of delivery, and the number of clicks that have been generated/detected may indicate the stage of delivery and/or the amount of medicament already delivered or still remaining.

Sensor 1052 may include piezoelectric transducers (e.g., PZT ceramic or MEMS) and/or single crystal materials. One or more sensors 1052 may be positioned on any suitable location of delivery device 1000 in a number of configurations. For example, in the embodiment depicted in FIG. 12A, sensor 1052 may be in an annular configuration positioned on a proximal region of housing 1021 within shell 1022. In the embodiment depicted in FIG. 12B, sensor 1052 may be positioned along sawtooth region 1025, between body 1020 and housing 1021. Sensor 1052 may circumscribe body 1020, or multiple discrete sensors 1052 can be positioned circumferentially and/or longitudinally along sawtooth region 1025.

Sensor 1052 may detect the clicks and/or vibrations, and the information detected may be processed or interpreted, for example, to generate an output indicative of the start, stop, speed, timing, position, displacement, and/or progress of a dosage delivery. The signals detected by sensor 1052 may be recorded, stored, processed to assess this information. For example, if the rate of clicks or volume of clicks is too high or too low, this may indicate that the medicament is being delivered at the wrong speed or that there may be a blockage in delivery device 1000. The initiation of clicks may indicate the commencement of delivery, and the number of clicks detected after initiation may indicate the progress of delivery. There may be a pre-determined number of clicks that indicates delivery completion, and if the clicks cease before this number is achieved, that may indicate that an incomplete dosage was delivered. Achieving the pre-determined number of clicks or the absence of clicks may indicate the end of dosage delivery.

The clicks and/or vibrations detected by sensor 1052 may be output as raw data or may be processed and then output. This data may be output in any manner as described herein. For example, one or more indicators 1056 may be positioned anywhere on delivery device 1000. In embodiments in which indicator 1056 is visual, indicator 1056 may be located on an exterior surface or a region visible from an exterior surface. For example, in the embodiment shown in FIG. 12A, indicator 1056 may be positioned on or within shell 1022, and indicator 1056 may be configured to illuminate, causing shell 1022 to glow. Output from indicator 1056 may convey to a user any of the information regarding, e.g., the progress, start, stop, speed, position, displacement, dosage amount, or any fault alerts (e.g., premature cessation of dosage delivery, or incorrect speed or timing) may be conveyed in any of the manners described herein. By integrating piezoelectric sensor system 1050 and indicator 1056 together in delivery device 1000, information may be gleaned and communicated to a user efficiently and easily.

While the present invention is described herein with reference to illustrative embodiments for particular applications, it should be understood that the invention is not limited thereto. Those having ordinary skill in the art and access to the teachings provided herein will recognize additional modifications, applications, embodiments, and substitution of equivalents all fall within the scope of the invention. For example, any type of indicator (visual, audio, tactile, etc.) may be used with any transponder system in any number or combination. Transponders may be used to communicate with any number of devices or indicators located on the delivery device, external to the delivery device, or any combination thereof via wired or wireless connections. In exemplary embodiments of the delivery device, a housing may be permanently or removeably attached to the syringe body or may be integrated as part the syringe body or may not be included. Components of the transponder may be in any arrangement or configuration. The transponders of present embodiments may be used and incorporated into off-the-shelf dose delivery systems or may be part of a unique dose delivery system. Accordingly, the invention is not to be considered as limited by the foregoing description.

What is claimed is:

1. A transponder for a dose delivery device for determining a characteristic of dose delivery, the transponder comprising:
a first sensor configured to detect at least a first parameter of the dose delivery device based on movement of at least one of the dose delivery device or a medicament contained within the dose delivery device, wherein the first sensor is configured to generate a first signal indicative of at least the first parameter;
a processor operably coupled to the first sensor and configured to process the first signal;
an indicator unit operably coupled to the processor and configured to generate an output based on the first signal, wherein the output is perceivable by a user; and
a battery configured to provide power to at least one of the first sensor, the processor, and the indicator unit.

2. The transponder of claim 1, wherein the processor is configured to determine whether the first parameter falls inside of or outside of a predetermined range.

3. The transponder of claim 1, wherein the transponder is configured to be formed as part of the dose delivery device.

4. The transponder of claim 1, wherein the transponder is configured to fit between a push rod and a plunger of the dosage delivery device.

5. The transponder of claim 1, wherein the transponder is a stand-alone component dimensioned to fit inside of the dose delivery device.

6. The transponder of claim 1, wherein the indicator unit is configured to be coupled to an external portion of the dose delivery device at a location separate from the location of the first sensor.

7. The transponder of claim 1, wherein the processor is configured to communicate with a receiver located external to the transponder.

8. The transponder of claim 7, wherein the receiver is configured to be located separate from the dose delivery device.

9. The transponder of claim 7, wherein the receiver is operably coupled to the indicator unit, and the indicator unit and the receiver are configured to be located at a location different than a location of the first sensor.

10. The transponder of claim 1, wherein the output from the indicator unit is visual.

11. The transponder of claim 1, wherein the first parameter includes at least one of a pressure, a speed, an acceleration, a time, a relative displacement, and a position.

12. The transponder of claim 1, wherein the first parameter is indicative of at least one of a start of the dose delivery, an end of the dose delivery, or progress of the dose delivery.

13. The transponder of claim 1, wherein the first sensor includes one of a pressure sensor, a strain gauge, and a switch, configured to generate the first signal indicative of a pressure applied to the push rod.

14. The transponder of claim 1, wherein the first signal is generated relative to a threshold.

15. The transponder of claim 1, further comprising a second sensor configured to generate a second signal based on a second parameter, wherein the processor is configured to process the second signal in addition to the first signal.

16. The transponder of claim 15, wherein the second sensor includes a temperature gauge configured to generate the second signal, and the second signal is indicative of a temperature of the medicament.

17. The transponder of claim 15, wherein at least one of the first sensor or the second sensor includes at least one of an accelerometer or a gyroscope, configured to determine the relative positioning of the transponder.

18. A dose delivery device having a transponder for determining a characteristic of the dose delivery device when delivering a dose of medicament, the dose delivery device comprising:
a body housing a reservoir within the body for containing the medicament;
an actuator configured for engagement by a user;
a push rod coupled to the actuator and extending through the body, the push rod configured to dispense the medicament from the reservoir;
a plunger located distal to the push rod and located adjacent to the reservoir; and
the transponder positioned within the body, the transponder comprising:
a sensor configured to detect at least one parameter of the dose delivery device and generate a signal indicative of the parameter;
a processor operably coupled to the sensor and configured to process the signal; and
an indicator unit operably coupled to the processor and configured to generate an output based on the signal, wherein the output is perceivable by a user.

19. The dose delivery device of claim 18, wherein the transponder is positioned within the body at a location proximal to the reservoir.

20. The dose delivery device of claim 18, wherein the sensor includes at least one of a pressure sensor, a strain gauge, or a switch, and the signal is indicative of a pressure applied to the push rod.

21. The dose delivery device of claim 18, wherein the sensor includes a temperature gauge, and the signal is indicative of a temperature of the medicament.

22. The dose delivery device of claim 18, wherein the sensor includes one of an accelerometer or a gyroscope, configured to determine the relative positioning of the transponder.

23. The dose delivery device of claim 18, wherein the dose delivery device is a syringe.

24. The dose delivery device of claim 18, wherein a distal end of the push rod abuts a proximal end of the sensor.

25. A dose delivery system comprising:
a dose delivery device, including:
a body having a reservoir located within a distal region of the body, wherein the reservoir is configured to contain a medicament;
a push rod having a plunger located at a distal end of the push rod, wherein the plunger defines a proximal end of the reservoir, and wherein the push rod and the plunger are cooperatively configured to dispense the medicament from the reservoir;
an actuator operably connected to a proximal end of the push rod and configured to advance the push rod in a distal direction through the body; and
a cannula attached to the distal end of the body through which the medicament from the reservoir is dispensed; and
a transponder, including:
a sensor dimensioned to be positioned within the body and configured to detect at least one parameter of a dose of the medicament being dispensed from the reservoir, wherein the sensor is configured to generate a signal indicative of the parameter;
a processor operably coupled to the sensor and configured to process the signal;
an indicator unit operably coupled to the processor and configured to generate an output based on the signal, wherein the output is perceivable by a user; and
a battery configured to provide power for at least one of the sensor, the processor, and the indicator unit.

26. The dose delivery system of claim 25, further comprising a wireless transmitter configured to communicate with a device located external to the dose delivery device.
